(19) United States
(12) Patent Application Publication
(10) Pub. No.: US 2004/0037879 A1
(43) Pub. Date: Feb. 26, 2004

(54) Oral controlled release forms useful for reducing or preventing nicotine cravings

(76) Inventors: Prasad S. Adusumilli, Parsippany, NJ (US); Cuong Quoc An, Warren, NJ (US); Shing Yue Chan, Parsippany, NJ (US); John Jiangnan Liu, Parsippany, NJ (US)

Correspondence Address: GLAXOSMITHKLINE, Corporate Intellectual Property - UW2220, P.O. Box 1539, King of Prussia, PA, 19406-0939, US

(21) Appl. No.: 10/278,213

(22) Filed: Oct. 21, 2002

Related U.S. Application Data
(60) Provisional application No. 60/336,353, filed on Nov. 2, 2001.

(51) Int. Cl. A61K 9/52; A61K 9/22

(52) U.S. Cl. 424/468; 424/457

(57) ABSTRACT

The present invention provides new oral dosage formulations comprising a nicotine active, optionally combined with an antidepressant, which through the controlled release of the active ingredient(s) alleviate some of the nicotine withdrawal symptoms a person may experience during attempts to quit smoking.

This application claims the benefit of U.S. Provisional Application No. 60/336,353, filed Nov. 2, 2001.

### BACKGROUND OF THE INVENTION

It is generally known that active as well as passive smoking of tobacco products, such as cigarettes, cigars, and pipe tobacco, presents serious health risks to the user and those subjected to secondary smoke. It is also known that use of other forms of tobacco, such as chewing tobacco, presents serious health risks to the user. Furthermore, the use of tobacco products in public areas is increasingly either restricted or socially unacceptable.

It is also recognized that reducing or quitting tobacco use is often very difficult for persons accustomed to using tobacco. This difficulty arises in large part from the addictive nature of nicotine. Efforts have therefore been made to provide nicotine substitutes to satisfy a tobacco user's cravings, but which avoid health risks associated with tobacco use, especially smoking.

In recent years, nicotine replacement therapies (NRT) have been successfully commercialized as a means to reduce or quit smoking or other forms of tobacco usage. Such commercial NRT include nicotine gums (e.g., NICORETTE) and nicotine transdermal patches (e.g., NICODERM).

In addition, nicotine lozenges have been marketed outside of the United States, for example, as STOPPERS and NICOTINELL brand lozenges. As far as the present inventors are aware, such lozenges are in the form of compressed tablets. In addition, U.S. Pat. Nos. 5,593,684, 5,721,257 and 5,362,496 (Baker et al.) disclose methods and therapeutic systems for smoking cessation, utilizing transdermal nicotine delivery for obtaining base-line nicotine plasma levels, coupled with transmucosal administration of nicotine to satisfy transient craving. One preferred transmucosal delivery system is a lozenge for buccal delivery, comprising nicotine dispersed in an adsorbent or absorbent excipient and a nonnutritive sweetener, preferably made by direct compression.

While such means are useful as aids to reduce or quit smoking, there is an ongoing need to provide improved or alternate NRT. For example, users may prefer to use forms other than chewing gum or transdermal patches. Certain users may dislike or be unable to chew gum, and users may desire more rapid craving relief than typically provided by transdermal patches. There is a need in the art, therefore, to develop nicotine-containing oral controlled release formulations which provide a rapid onset followed by a prolonged effect in order to reduce or prevent nicotine cravings and overcome the deficiencies of the current state of the art.

### SUMMARY OF THE INVENTION

The present invention provides new orally administerable dosage formulations comprising a nicotine active. The oral dosage form allows for the release of nicotine in the oral and/or buccal cavity thereby providing a rapid rise in blood plasma nicotine levels, followed by a delivery of nicotine in the GI tract for a prolonged maintenance of nicotine levels in the blood in order to reduce or prevent nicotine cravings. The nicotine active may optionally be combined with an antidepressant or antianxiolytic in order to alleviate some of the nicotine withdrawal symptoms a person may experience during attempts to quit smoking. Novel nicotine formulations for the delivery of nicotine to the stomach and intestines are also disclosed.

### BRIEF DESCRIPTION OF THE DRAWINGS

FIG. 1 is a schematic drawing showing the various layers of the controlled release formulation.

FIG. 2 is a graph showing the % release of nicotine in phosphate buffer (pH 7.4) from a formulation prepared in accordance with Example 1.

FIG. 3 is a schematic drawing showing the cross-section of a controlled release formulation made using a dry coating process.

FIG. 4 is a schematic drawing showing the cross-section of a capsule containing beads.

FIG. 5 is a schematic drawing showing controlled release capsule containing beads.

FIG. 6 is a schematic drawing showing a gelatin enrobed controlled release nicotine core.

### DETAILED DESCRIPTION

The present invention is directed to new controlled release oral dosage formulations having oral and GI tract release characteristics, which may take the form of controlled release tablets and capsules containing pellets, beads, etc. comprising a nicotine active, optionally combined with an antidepressant or antianxiolytic, which can provide a release of a nicotine active in the oral and/or buccal cavity directly upon administration, followed by a release of nicotine active once the oral dosage form reaches the gastrointestinal system. Hereinafter it should be understood that oral release refers to the immediate release of nicotine to the oral and/or buccal cavity, to be distinguished from nicotine release in the GI tract which may be immediate release, sustained release or both.

The oral dosage formulations of the present invention may be in solid form or may be used in conjunction with any multiparticulate system, such as granules, spheroids, beads, pellets, ion-exchange resin beads, and other multiparticulate systems in order to obtain a desired sustained-release of the nicotine active. Beads, pellets, granules, spheroids, or pellets, etc., prepared in accordance with the present invention can be presented in a capsule or in any other suitable unit dosage form. An amount of the multiparticulates effective to provide the desired dose of drug over time may be placed in a capsule, or may be incorporated in any other suitable oral solid form, such as a tablet. With respect to all such optional formulations, it is desired that the formulation be prepared such that it provides an initial immediate release of nicotine while the formulation is in the oral cavity, which release is analogous to an immediate release formulation, and that the formulation further provides a GI tract release component which delivers and maintains therapeutically effective levels of nicotine in the plasma for the desired amount of time once it reaches the gastrointestinal system, preferably the intestine. The oral release component preferably represents from about 0.1% to about 15% of the total dose and the GI tract release component preferably represents from about 85% to 99.9% of the total dose of nicotine active contained in the formulations of the present invention. In certain preferred embodiments, the oral release component represents about 2-4% of the total dose and the GI tract release component represents about 96-98% of the total dose of nicotine active contained in the formulation.

In a preferred embodiment the novel controlled release form is a solid formulation such as a tablet or pill. Thus, in its broadest aspects the present tablet would include a gastrointestinal (GI) tract release core and an oral release layer overlying the core. As mentioned above, the oral release layer provides immediate release of nicotine to the oral and/or buccal cavity to quickly satisfy the nicotine cravings of a user. The GI tract release core may conveniently release nicotine into the GI tract to provide for a continued and sustained supply of nicotine over a longer period of time. Thus, in use, the solid dosage form of the present invention needs to be retained in the mouth for a certain period of time sufficient to deliver the desired dose of nicotine to the oral and/or buccal cavity. Thereafter, the solid dosage form is swallowed to continue with the next stage of nicotine delivery to the GI tract.

The GI tract release core may be designed to provide a number of different options for delivery of nicotine including

a) immediate release in the stomach; or

b) immediate release in intestines; or

c) sustained release in intestines; or

d) immediate release in stomach, followed by either immediate or sustained release in intestines.

In accordance with the present invention, each of these options (a)-(d) would be designed into a single oral dosage form which also provides an immediate release of nicotine to the oral and/or buccal cavity, as mentioned above. In the case of option (d) above, the novel formulation may therefore comprise an oral release component and a two-part GI tract release component. This two-part GI tract release component would comprise an intestinal release core for the sustained or immediate release of nicotine to the intestines, and a stomach release layer overlying the intestinal release core for the immediate release of nicotine to the stomach . It should be understood that “sustained or immediate release in the intestines” may encompass nicotine delivery to the small intestine, the large intestine or both. Preferably, an enteric coating layer is interposed the stomach release layer and intestinal release core. It will be appreciated by those skilled in the art that the description below of materials suitable for the GI tract component or GI tract core would be applicable to the preparation of this two-part GI tract component as well.

Further, this two-part GI tract release form is a novel nicotine delivery formulation in its own right and, accordingly, is considered a part of the present invention, even without an oral release component.

Optionally, the solid dosage forms of the present invention may include other layers or portions to accomplish or enhance the desired release characteristics referred to above. For example, it is desirable to include buffer agents in or near the oral release layer in order to bring the saliva in the oral/buccal cavity nearer to the pH 7-10 range for enhanced absorption of nicotine across the orali/buccal mucosa. Accordingly, a buffer layer may be either over or under the oral release layer. Also, depending upon the buffers and source of nicotine used, it may be advantageous to employ a physical barrier layer between the oral release layer and the buffer layer. Further, for those situations where it is desired that the GI tract release core only delivers nicotine to the intestines, then an enteric coating on the core could be utilized.

The oral release layer comprises a film formed of one or more water soluble polymers, one or more plasticizers, a source of nicotine and, of course, small amounts of the solvent, e.g. water, used in processing. Water soluble polymers found useful in the present invention are hydrophilic polymers and polysaccharides, and alkylcellulose polymers.

Hydrophylic polymers and polysaccharides suitable for use in the oral release layer include sodium carboxymethylcellulose, partially cross-linked polyacrylic acid [??], hydroxyethylcellulose, hydroxypropylcellulose, hydroxypropylmethylcellulose (HPMC), polyethylene oxide, pectin, gelatin, calcium silicate, starches, gums, e.g., xanthan gum, locust bean gum, guar gum, gum acacia, gum arabic or a mixture thereof. The most preferred hydrophylic polymer is HPMC (Dow Chemical Company) having molecular weight of between 3,000-100,000.

Cellulosic materials and polymers, including alkylcelluloses provide hydrophobic materials suitable for forming the oral release layer or for coating the beads according to the invention. Simply by way of example, one preferred alkylcellulosic polymer is ethylcellulose, although the artisan will appreciate that other cellulose and/or alkylcellulose polymers may be readily employed, singly or in combination, as all or part of a hydrophobic coating according to the invention.

One commercially available aqueous dispersion of ethylcellulose is Aquacoat® (FMC Corp., Philadelphia, Pa., U.S.A.). Aquacoat® is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the Aquacoat® with a suitable plasticizer to use.

Another aqueous dispersion of ethylcellulose is commercially available as Surelease® (Colorcon, Inc., West Point, Pa., U.S.A.). This product is prepared by incorporating plasticizer into the dispersion during the manufacturing process. A hot melt of a polymer, plasticizer (dibutyl sebacate), and stabilizer (oleic acid) is prepared as a homogenous mixture, which is then diluted with an alkaline solution to obtain an aqueous dispersion which can be applied onto substrates.

Suitable plasticizers for the oral release layer include polyethylene glycol (PEG), propylene glycol, mineral and vegetable oils, and TriEthylAcetate. Generally, the amount of plasticizer included is based on the concentration of the film-former, e.g., most often from about 1 to about 10 percent by weight of the film-former. Concentration of the plasticizer, however, can only be properly determined after careful experimentation with the particular solution and method of application.

Plasticizers particularly suited for ethylcellulose-based oral release layers include water insoluble plasticizers such as dibutyl sebacate, diethyl phthalate, triethylcitrate, tributyl citrate, and triacetin, although it is possible that other water-insoluble plasticizers (such as acetylated monoglycerides, phthalate esters, caster oil, etc.) may be used. Triethyl citrate is an especially preferred plasticizer for the aqueous dispersions of ethyl cellulose of the present invention.

The nicotine active in the formulations of the invention may be selected from a wide variety of nicotine sources such as pharmaceutically acceptable salts of nicotine. Non-limiting examples of such salts include nicotine monotartrate (Spectrum Chemical Mfg. Corp. Simex, Seigfriend 2H20 CBC (America) Corp.) and bitartrate, nicotine hydrochloride, nicotine dihydrochloride, nicotine sulfate, nicotine zinc chloride monohydrate and nicotine salicylate. (FTL International Inc., Seigfriend Interchem Corp.) Nicotine oil and nicotine polacrilex are also potential nicotine sources. Nicotine polacrilex are nicotine-ion-exchange resin complexes, and are commercially available from Nicobrand.

Preferably, the oral release layer is a film overlying the GI tract release core and comprises HPMC 5cps or HPMC 15cps in an amount of from about 6-20% by weight of the oral release layers. This preferred oral release layer further comprises about 0.02 to 0.06% of a plasticizer selected from polyethylene glycol, propylene glycol, mineral and vegetable oil and triethylacetate along with nicotine active sufficient for the immediate oral/buccal dosage which can be 0.1 to 0.2% by weight of the oral release layer.

As mentioned above, it may be desirable to employ buffering agents sufficient to provide that the saliva is in the pH 7-10 range. This can be accomplished by employing a separate buffer layer or by incorporating buffering agents directly into the oral release layer.

Buffering agents contemplated by the present invention include sodium carbonate sodium bicarbonate, sodium hydroxide, sodium phosphate, calcium carbonate, magnesium carbonate, magnesium hydroxide, potassium hydroxide, aluminum hydroxide, as well as combinations of the above mentioned buffering agents. When the buffering agents are utilized and are to be incorporated into the oral release layer, they can be incorporated in any convenient amount designed to provide the desired pH of the saliva. Typically, an oral release layer may include from about 0.5 to about 3.0 mg/tablet of buffer material in the oral release layer, but it should be understood that more or less could be used depending upon the endpoint desired for saliva pH.

The GI tract release core comprises a source of nicotine sufficient to provide from about 1-60 mg equivalent of nicotine free base, and optionally an antidepressant or antianxiolytic, combined with one or more polymers which are selected according to the desired release characteristics within the GI tract. For example, if an immediate release of nicotine in the stomach is desired, the core may comprise one or more polymers selected from hydrophilic polymers and polysaccharides, and alkylcellulose polymers as discussed above, and further may include a plasticizer, all as discussed above regarding the oral release layer.

When it is desired to provide a sustained release of nicotine in the stomach and/or intestines, the GI tract release core comprises the nicotine active, optional antidepressant, and one or more polymers selected from hydrophilic polymers and polysaccharides, alkylcellulose polymers, and acrylic polymers.

The polymers are typically present in a range of up to about 80% and more preferably in the range of about 5 to 50% of the GI tract release core. Preferred polymers include sodium carboxymethylcellulose, partially cross-linked polyacrylic acid, hydroxyethylcellulose, hydroxypropylcellulose, polyethylene oxide, pectin, gelatin, or a mixture thereof. The most preferred hydrophylic polymer is HPMC having molecular weight of between 3,000-100,000. The GI tract release core may further comprise fillers in the range of about 10-80% and most preferably in the amount of 30-60% of the core. Fillers may be any convenient filler known in the art and non-limiting examples include dicalcium phosphate, pregelatinized starch, lactose spray dry, sorbitol, mannitol, microcrystalline cellulose, alone or in combination. The most preferred fillers are dicalcium phosphate and pregelatinized starch, alone or in combination. The GI tract release core may further comprise lubricants, non-limiting examples of which include magnesium stearate, stearic acid, vegetable oil, talcum, starch, mineral oil and PRUV®. The most preferred lubricants are magnesium stearate and stearic acid, alone or in combination. The amount of lubricants in the core range from about 0.5-10% and most preferably in the amount of 1-4%. Optionally, the core may include a glidant, such as silicon dioxide, corn starch, or talcum, alone or in combination, in a range of about 0.5-10% and most preferably in the amount of 0.5-1.0% of the core. The most preferred glidant is silicon dioxide.

For nicotine release in the intestine it is preferred to employ an enteric coating overlying the GI tract release core, that is, a coating which substantially prevents dissolution in the stomach. The enteric coating is preferably one or more acrylic polymers and may also include one or more plasticizers.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin.

Any antidepressants or antianxiolytics may be employed in the present invention, either alone or in combination. Antidepressants may be tricyclic antidepressants such as amitriptyline and nortriptyline or preferably are selective serotonin re-uptake inhibitors such as fluoxetine, sertraline, citaloprolam, fluvoxamine, pararetine, and bupropion. These should be included in known therapeutically effective amounts.

The hydrophobic material for the enteric coating may comprise any known pharmaceutically acceptable material which resists dissolution in the stomach. Preferably the enteric coating polymer may comprise a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers. The enteric coat may further comprise fillers, plasticizers and other materials as is known in the art.

In certain preferred embodiments, the acrylic polymer is comprised of one or more ammonia methacrylate copolymers. Ammonia metbacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonia methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. The term “pH-dependent” for purposes of the present invention is defined as having characteristics (e.g. dissolution) which vary according to environmental pH (e.g., due to changes in the in-vitro dissolution media, or due to passage of the dosage form through the gastrointestinal tract.

For example, there are a family of copolymers synthesized from diethylaminoethyl methacrylate and other neutral methacrylic esters, also known as methacrylic acid copolymer or polymeric methacrylates, commercially available as Eudragit® from Rohm Tech, Inc. There are several different types of Eudragit®. For example, Eudragit® E is an example of a methacrylic acid copolymer which swells and dissolves in acidic media. Eudragit® L is a methacrylic acid copolymer which does not swell at about pH<5.7 and is soluble at about pH>6. Eudragit® S does not swell at about pH<6.5 and is soluble at about pH>7. Eudragit® RL and Eudragit® RS are water swellable, and the amount of water absorbed by these polymers is pH-dependent, however, dosage forms coated with Eudragit® RL and RS are pH-independent. The term “pH-independent” for purposes of the present invention is defined as having characteristics (e.g., dissolution) which are substantially unaffected by pH, in that a difference, at any time, between an amount of methylphenidate released at one pH and an amount released at any other pH, when measured in-vitro using the USP Paddle Method of U.S. Pharmacopeia XXII (1990) at 100 rpm in 900 ml aqueous buffer, is no greater than 10%.

In certain preferred embodiments, the acrylic coating comprises a mixture of two acrylic resin lacquers commercially available from Rohm Pharma under the Tradenames RL30D and Eudragit® RS30D, respectively. Eudragit® RL30D and Eudragit® RS30D are copolymers of acrylic and methacrylic esters with a low content of quaternary ammonium groups, the molar ration ammonium groups to the remaining neutral (meth)acrylic esters bing 1:20 in Eudragit® RL30D and 1:40 in Eudragit® RS30D. The mean molecular weight is about 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids.

The Eudragit® RL/RS dispersions of the present invention may be mixed together in any desired ratio in order to ultimately obtain a sustained release formulation having a desirable dissolution profile. Desirable sustained release formulations may be obtained, for instance, from a retardant coating derived from 100% Eudragit® RL, 50% Eudragit RL and 50% Eudrait® RS, and 10% Eudragit® RL: 90% Eudragit® RS. Of course, one skilled in the art will recognize that other acrylic polymers may also be used, such as, for example, Eudragit® L.

As mentioned above, for cases where it is preferable to keep buffering agents separate from the sources of nicotine it will be advantageous to employ a buffer barrier layer which can be the outermost layer on the tablet or pill. This buffer layer comprises material containing a buffer or buffers raising the pH of the saliva from 5.6-7.6 to 8 so the nicotine active is converted for easy absorption though the oral membrane. For example, raising the pH of the saliva to 8-9 allows for the conversion of nicotine salt to nicotine base. The alkaline media is necessary to convert the nicotine salt to nicotine free base for easier absorption. Preferably, the buffer layer would have a pH of 8-11. Preferred buffer agents include sodium carbonate, sodium bicarbonate, sodium hydroxide, sodium phosphate, calcium carbonate, magnesium carbonate, magnesium hydroxide, potassium hydroxide, aluminum hydroxide, as well as combinations of the above mentioned buffer agents. The most preferred buffer agent is sodium carbonate. Preferably, the buffer agent would comprise about 0.01-6% and most preferably about 0.1-0.3% of the outer buffer barrier layer. The other ingredients comprising the buffer barrier layer include film forming substances, e.g. hydrophilic polymers and polysaccharides and/or alkylcellulose polymers, such as HPMC 5cps, HPMC 15cps, comprising about 6-20% of the outer buffer barrier layer; plasticizer, such as polyethylene glycol (PEG), propylene glycol, mineral, vegetable oils, and TriEthylAcetate, comprising about 0.2-0.06% of the outer buffer barrier layer; and water used in dissolving the materials during the manufacturing step.

It may also be advantageous to employ a physical barrier layer interposed with the buffer layer and the oral release layer to further isolate the nicotine active from the buffers. In such a case, a physical barrier layer formed as above for the buffer layer, but without the buffer agents, can be employed.

Lubricants, as mentioned above, include magnesium stearate, stearic acid, vegetable oil, talcum, starch, mineral oil, PRU®, and mixtures thereof.

Glidants used in the present invention include silicon dioxide, corn starch spray dried lactose, pregelatinized starch and talcum alone or in combination. Fillers used in the present invention include dicalcium phosphate, pregelatinized starch, lactose spray dry, sorbitol, mannitol, microcrystalline cellulose, acacia gum, gum arabic and other pharmacologically inert materials commonly used in pharmaceutical formulations and mixtures thereof.

Other ingredients to enhance stability, absorption, flavor, taste, and mouth fresheners may also be added to the formulation. such ingredients include: antioxidants such as butyl-hydroxy toluene and tocopherols and its salts, Vitamin E, absorption enhancers such as surfactants, alpha cyclodextrin, beta cyclodextrin, gamma cyclodextrin as well as other derivatives of cyclodextrin; flavor to mask the nicotine taste and mouth freshener, such as mint, menthol, pepper, tobacco, cinnamon, peppermint, spearmint, anise and eucalyptus; and nutritive and nonnutritive (for health benefits to diabetics or to reduce calorie uptake) sweeteners. Other ingredients such as tooth whiteners, anti-tooth decay compounds, antibacterial compounds and preservatives may also be added and are also contemplated by the present invention.

In one preferred embodiment, the present invention provides a controlled release solid form formulation, such as a tablet, containing a series of layers with different characteristics including a buffer barrier layer, a physical barrier layer, an oral release layer, and a GI tract release core with an enteric coating. Optionally, the solid form formulation may also contain a buffer layer between the oral release layer and the enteric coat. The solid form formulation is designed such that upon oral administration, the formulation provides a rapid dissolution and absorption of the outer layers including the oral release layer which contains a portion of the nicotine active in immediate release form, thereby resulting in a rapid rise of the drug to therapeutic plasma levels while the solid oral formulation is still in the oral cavity. This is followed by a period of no absorption while the solid oral formulation travels through the stomach, followed thereafter by a sustained release of the drug from the formulation to achieve desired plasma levels once the core of the solid oral formulation reaches the intestine. The solid form formulation may be placed in the oral cavity or under the tongue for the dissolution and/or absorption of the outer layers, including the oral release layer. Subsequently, the core, with its remaining layers, such as the enteric coating and the optional buffer layer, may be swallowed with or without the aid of water. In another preferred embodiment, the present invention provides beads encapsulated within a capsule wherein the capsule is impregnated or covered with a nicotine active and other pH altering and/or flavoring ingredients. Each bead within the capsule contains a GI tract release core. Optionally, each bead may contain an enteric coat or a buffer layer on the GI tract release core. The formulation is designed such that upon oral administration, the formulation provides a rapid dissolution of nicotine from the capsule impregnated or coated with nicotine active, thereby resulting in a rapid rise of the drug to therapeutic plasma levels while the formulation is still in the oral cavity. This followed by a period of low absorption (if no enteric coat is employed) or no absorption (if the enteric coat is included) while the formulation travels through the stomach, followed thereafter by a controlled release of the drug from the beads to maintain plasma levels once they reach the intestine. The capsule may be placed in the oral cavity for the absorption of the nicotine. Subsequently, the capsule may be swallowed with or without the aid of water. Optionally, the nicotine impregnated or covered capsule may be designed to dissolve completely in the oral cavity and allowing the user to swallow the pellets only. With such a capsule, the beads may be enteric coated in order to maintain the beads intact until they reach the intestine.

In other embodiments of the invention, the bead size and the drug release profiles from the formulations can be adjusted in order to obtain a desired pharmacokinetic profile.

In another preferred embodiment, a solid form formulation comprises an outer buffer barrier layer, a physical barrier layer, an immediate release layer containing 0.25 mg to 1.0 mg nicotine base to curb the cigarette craving within 1- 10 minutes of intake, and an inner sustained release core containing 20-60 mg nicotine base capable of elevating nicotine blood plasma levels for a period of 6 to 24 hours. Optionally, the solid form formulation may contain an enteric coat between the immediate release layer and the inner sustained release core. Optionally, the solid form formulation may contain a buffer layer before the immediate release layer and the enteric coat, or, in the absence of an enteric coat, before the immediate release layer and inner sustained release core.

The present invention is further directed to oral controlled release formulations which combine both a rapid onset and sustained plasma concentrations throughout the day, followed by a drop-off of plasma concentrations of nicotine to below minimum effective concentrations during sleeping period to minimize any sleep disturbances associated with elevated nicotine level.

In certain preferred embodiments, the formulation provides a time to maximum plasma concentration of nicotine active in about 2 minutes to 30 minutes after oral administration and provides effective blood levels for at least about 4 hours after administration depending on the sustained released formulations used.

The formulations of the present invention are designed to produce a rapid rise to therapeutic plasma levels after oral absorption, due to the rapid dissolution and absorption of the outer layer)s), followed by a period of reduced absorption after controlled release of the nicotine from the GI tract release component, to maintain therapeutic plasma levels. After absorption of nicotine released from the oral release component, plasma levels would then decrease according to the elimination kinetics of the drug.

Tablets, (spheroids, beads etc.) made of the formulation of the GI tract release core are prepared by screening the ingredients through an appropriately sized screen, mixing the ingredients and comprising the resulting mixture to a hardness of about 5 to 20 SCU. Compressing machines well known in the art, such as Stoke F3, can be used to form the sustained release core. Spheroids or beads coated with the formulation of the GI tract release core are prepared, e.g., by dissolving the therapeutically active agent in water and then spraying the solution onto a substrate, for example, Nu Pariel® 18/20 beads, using a Wurster insert. Optionally, additional ingredients are also added prior to coating the beads in order to assist the binding of the drug to the beads, and/or to color the solution, etc. For example, a product which includes hydroxypropylmethylcellulose, etc. with or without colorant (e.g., Opadry®, commercially available from Colorcon, Inc.) may be added to the solution and the solution mixed (e.g., for about 1 hour) prior to application of the same onto the beads. The resultant coated substrate, in this example beads, may then be optionally overcoated with a buffer agent, to separate the therapeutically active agent from the enteric coating. An example of a suitable barrier agent is one which comprises hydroxypropylmethylcellulose. However, any film-former known in the art may be used. It is preferred that the barrier agent does not affect the dissolution rate of the final product.

The beads may then be overcoated with an aqueous dispersion of the hydrophobic material. The aqueous dispersion of hydrophobic material preferably further includes an effective amount of plasticizer, e.g., triethyl citrate. Pre-formulated aqueous dispersions of ethylcellulose, such as Aquacoat® or Surelease®, may be used. If Surelease® is used, it is not necessary to separately add a plasticizer. Alternatively, pre-formulated aqueous dispersions of acrylic polymers such as Eudragit® can be used.

The enteric coating solutions of the present invention preferably contain, in addition to the film-former, plasticizer, and solvent system (i.e., water), a colorant to provide elegance and product distinction. Color may be added to the solution of the therapeutically active agent instead, or in addition to the aqueous dispersion of hydrophobic material. For example, color may be added to Aquacoat® via the use of water based, alcohol or propylene glycol based color dispersions, milled aluminum lakes and opacifiers such as titanium dioxide by adding color with shear to water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used to include titanium dioxide and color pigments, such as iron oxide pigments. The incorporation of pigments, may, however, increase the retard effect of the coating.

In formulations where an aqueous dispersion of a hydrophobic polymer such as an alkylcellulose is applied to the substrate, it is preferred that the coated substrate is cured at a temperature above the glass transition temperature of the plasticized polymer and at a relative humidity above ambient conditions, until an endpoint is reached at which the coated formulation attains a dissolution profile which is substantially unaffected by exposure to storage conditions, e.g., of elevated temperature and/or humidity. Generally, in such formulations the curing time is about 24 hours or more, and the curing conditions may be, for example, at about 60° C. and 85% relative humidity. Detailed information concerning the stabilization of such formulations is set forth in U.S. Pat. Nos. 5,273,760, 5,681,585, and 5,472,712, all of which are hereby incorporated by reference in their entireties.

The sustained release profile of the coated formulations of the invention can be altered, for example, by varying the amount of overcoating with the aqueous dispersion of hydrophobic material, altering the manner in which the plasticizer is added to the aqueous dispersion of hydrophobic material, by varying the amount of plasticizer relative to hydrophobic material, by the inclusion of additional ingredients or excipients, by altering the method of manufacture, etc. The dissolution profile of the ultimate product may also be modified, for example, by increasing or decreasing the thickness of the enteric coating.

The plasticized aqueous dispersion of hydrophobic material may be applied onto the substrate comprising the therapeutically active agent by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the aqueous dispersion of hydrophobic material to obtain a predetermined sustained release of the therapeutically active agent (i.e., drug) when the coated substrate is exposed to aqueous solutions, e.g. gastric fluid, is preferably applied, taking into account the physical characteristics of the therapeutically active agent, the manner of incorporation of the plasticizer, etc. After coating with the hydrophobic material, a further overcoat of a film-former, such as Opadry, is optionally applied to the beads. This overcoat is provided, if at all, in order to substantially reduce agglomeration of the beads.

The release of the nicotine and/or antidepressant from the sustained release formulation of the present invention can be further influenced, i.e., adjusted to a desired rate, by the addition of one or more release-modifying agents, or by providing one or more passageways through the coating. The ratio of hydrophobic material to water soluble material is determined by, among other factors, the release rate required and the solubility characteristics of the material selected.

The release-modifying agents which function as pore-formers may be organic or inorganic, and include materials that can be dissolved, extracted or leached from the coating in the environment of use. The pore-formers may comprise one or more hydrophilic materials such as hydroxypropylmethylcellulose, methylcellulose.

The sustained release core of the present invention can also include erosion-promoting agents such as starch sr (Primcontrol)® and gums. The sustained release core of the present invention can also include materials useful for making microporous lamina in the environment of use, such as polycarbonates comprised of linear polyesters of carbonic acid in which carbonate groups reoccur in the polymer chain.

The release-modifying agent may also comprise a semi-permeable polymer. In certain preferred embodiments, the release-modifying agent is selected from hydroxyproplymethylcellulose, ethylcellulose or methecylate copolymers and mixtures of any of the foregoing.

### EXAMPLE 1

### Manufacture and Composition of a Controlled Release Tablet (Wet Coat)

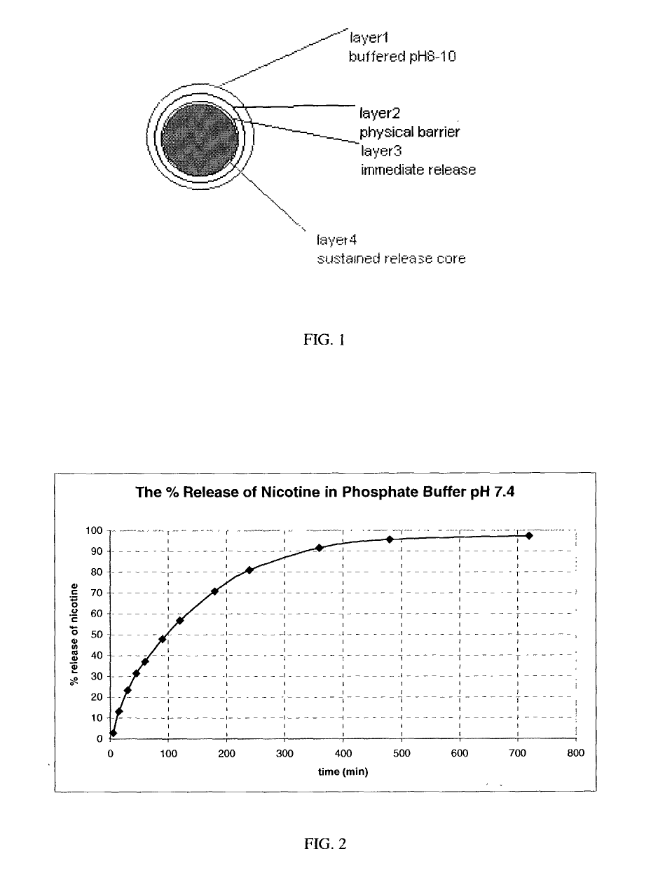

A schematic of the layers appear in FIG. 1.

### EXAMPLE 1(a)

### The GI Tract (Sustained) Release Core (Layer 4)

To form the inner core, all raw materials were screened through an appropriate screen and were mixed in a PK blender for 10 minutes at 10-15 RPM. Once the ingredients were mixed, the material was compressed into 220-240 mg tablet cores with a hardness of 4-10 SCU using a deep concave around 5/16″ punch and F3 press. The ingredients used to form the inner sustained release core of this example are listed in Table I below:

TABLE 1

| Ingredient | mg/core |
|---|---|
| Nicotine bitartrate | 76.00 |
| HPMC 15K | 63.25 |
| Lactose spray dry | 86.25 |
| Magnesium stearate | 4.20 |
| Silicon dioxide | 2.30 |
| Total | 232.00 |

### EXAMPLE 1(b)

### The Oral Release Layer (Layer 3)

A mixture of 1000 grams of approximately 10-12% solid (w/w) aqueous film solution was made with HPMC 5cpc, HPMC 15cpc and propylene glycol using the ratios set forth in Table 2. Subsequently, 6.5 grams of Nicotine bitartrate was added to 200 grams of film solution. A coating pan (Acela Cota 12″ diameter) was loaded with 1000 grams (the equivalent of 4310 tablets), of core from Example 1(a), and the film solution was sprayed at a rate of 6-8 grams per minute. The inlet temperature was 80-90 ° C. and the outlet temperature was 38-42° C. During the spraying the coating pan was rotated at 10-12 rpm. Spraying was continued until the film solution was finished (206.5 grams). The approximate weight gained after coating was around 3% w/w.

TABLE 2

| Ingredient | mg/tablet |
|---|---|
| HPMC 5 cps | 4.0 |
| HPMC 15 cps | 1.0 |
| Propylene glycol | 0.5 |
| Nicotine bitartrate | 1.5 |
| Water (evaporates during coating) | 0.0 |

### EXAMPLE 1(c)

### The Physical Barrier Layer (Layer 2)

A mixture of 1000 grams of approximately 10-12% solid (w/w) aqueous film solution was made with HPMC 5cpc, HPMC 15cpc and propylene glycol using the ratios set forth in Table 3. This aqueous film solution was subsequently sprayed on top of the immediate release layer of Example 1(b). About 100-200 grams of this neutral solution was used.

TABLE 3

| Ingredient | mg/tablet |
|---|---|
| HPMC 5 cps | 4.0 |
| HPMC 15 cps | 1.0 |
| Propylene glycol, | 0.5 |
| Water (evaporates during coating) | 0.0 |

### EXAMPLE 1(d)

### The Buffer Barrier Layer (Layer 1)

A mixture of 1000 grams of approximately 10-12% solid (w/w) aqueous film solution was made with HPMC 5cpc, HPMC 15cpc, propylene glycol, and sodium carbonate using the ratios set forth in Table 4, yielding a high pH solution (pH 8-10). This aqueous film solution was sprayed on top of the physical barrier layer of Example 1(c). About 100-200 grams of this high pH solution was used in this example.

TABLE 4

| Ingredient | Mg/tablet |
|---|---|
| HPMC 5 cps | 4.0 |
| HPMC 15 cps | 1.0 |
| Propylene glycol | 0.5 |
| Sodium carbonate | 1.5 |
| Water (evaporates during coating) | 0.0 |

### EXAMPLE 1(e)

To demonstrate the release characteristics of the nicotine dosage form of the present invention, the solid dosage form of this Example 1 was placed in a USP Apparatus type I (basket) and revolved at 100 RPM in a 37° C. (±0.5° C.) USP phosphate buffer solution pH 7.4 Samples were collected at 5, 15, 30, 45, 60 and 90 minutes and at 2, 3, 4, 6, 8 and 12 hours to measure the amount of nicotine released. The results are in FIG. 1 below

[image]

| % Nicotine release | Time (minutes) |
|---|---|
| 5 | 2.9 |
| 15 | 13.2 |
| 30 | 23.3 |
| 45 | 31.4 |
| 60 | 37.1 |
| 90 | 47.8 |
| 120 | 56.7 |
| 180 | 70.8 |
| 240 | 81 |
| 360 | 91.6 |
| 480 | 95.6 |
| 720 | 97.3 |

### EXAMPLE 2

### Manufacture and Composition of a Controlled Release Tablet (Dry Coat)

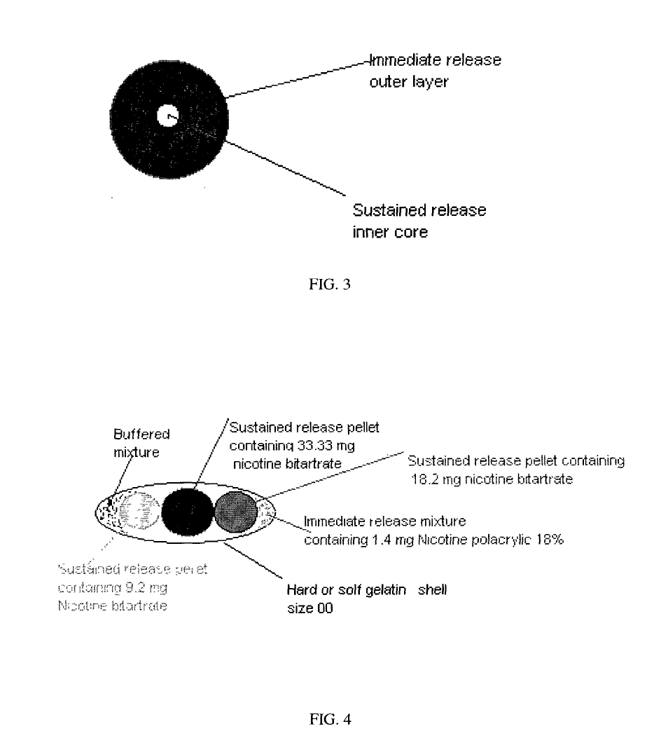

A schematic of the layers appears in FIG. 3.

### EXAMPLE 2(a)

### The GI Tract (Sustained) Release Core

The GI tract sustained release core was prepared according to Example 1(a), or Example 3 using pellets in table 8, 9 and 10 depending on the desired dissolution profile from 6 hours to 12 hours or longer.

### EXAMPLE 2(b)

### The Oral Release Layer, Using Quick Dissolving Technology for Fast Disintegration in the Oral Cavity

The oral release layer is composed of the ingredients listed in Table 5 below. Raw materials were screened through #20US screen except Sorbitol, Peppermint and Blue#2 were screened through a #50US mesh . All raw materials were mixed in a PK blender for 10 minutes at 10-15 RPM. Using an F3 single punch press, the punch die was hand filled with 1000 mg of immediate release blend. The sustained release core of Example 2(a) was deposited on top, in the middle on the oral release portion in the punch die, and was compressed resulting in a tablet having 2 portions with the GI tract sustained release core on the inside and the oral release portions forming the outer shell. A variation could be such that the outer layer completely encases the GI tract sustained release core.

TABLE 5

| Ingredient | mg/tablet |
|---|---|
| Nicotine bitartrate | 1.50 |
| Avicel CE-15 | 200 |
| Sodium carbonate | 10 |
| Sorbitol granular | 268 |
| Corspovidone XL-10 | 50 |
| Acesulfame K | 8 |
| Peppermint flavor | 26 |
| Mint citrus | 25 |
| FD&C Blue#2, 12-14% dye, A1. lake | 3 |
| Magnesium stearate | 8 |
| Total | 1000 |

### EXAMPLE 3

### Manufacture and Composition of a Controlled Release Capsule

A schematic of the controlled release capsule appears in FIG. 4.

Round pellets, size from 6-8 mm in diameter, were manufactured by mixing nicotine bitartrate into a polymer matrix to obtain a sustained release profile. The pellets have different strengths and dissolution profiles, e.g. type 1 pellet containing 33.33 mg Nicotine bitartrate corresponding to 11 mg Nicotine base, type 2 containing 18.2 mg Nicotine bitartrate corresponding to 6 mg Nicotine base, type 3 pellet containing 9.2 mg Nicotine bitartrate corresponding to 3 mg Nicotine base.

The composition of the buffered mixture, oral release mixture and pellets type 1,2 and 3 are listed in tables 6, 7, 8, 9, and 10.

TABLE 6

| Buffered Mixture | mg/capsule |
|---|---|
| Sodium carbonate | 5 |
| Mint citrus flavor | 15 |
| Sorbitol | 40 |
| Total | 60 |

TABLE 7

| Immediate release mixture | mg/capsule |
|---|---|
| Nicotine polacrilex 18% | 1.4 |
| Sorbitol | 75.6 |
| Total | 77.0 |

TABLE 8

| Pellet type 1 containing 11 mg nicotine base | mg/pellet | % |
|---|---|---|
| Nicotine bitartrate | 33.33 | 13.77 |
| HPMC 100M | 72 | 29.75 |
| Starch SR, Primcontrol ® | 50 | 20.66 |
| Starch pregelatinized | 81.67 | 33.75 |
| Mg. Stearate | 3 | 1.24 |
| Cabosil | 2 | 0.83 |
| Total | 242 | 100.00 |

TABLE 9

| Pellet type 2 containing 6 mg nicotine base | mg/pellet | % |
|---|---|---|
| Nicotine bitartrate | 18.2 | 7.55 |
| HPMC 100M | 67 | 27.80 |
| Starch pregelatinized | 82.8 | 34.36 |
| Starch SR, Primcontrol ® | 67 | 27.80 |
| Red Carmine | 1 | 0.41 |
| Mg. Stearate | 3 | 1.24 |
| Cabosil | 2 | 0.83 |
| Total | 241 | 100.00 |

TABLE 10

| Pellet type 3 containing 3 mg nicotine base | mg/pellet | % |
|---|---|---|
| Nicotine bitartrate | 9.2 | 4.18 |
| HPMC 100M | 68 | 30.91 |
| Starch pregelatinized | 69 | 31.36 |
| Starch SR, Primcontrol ® | 68 | 30.91 |
| FD&C blu#2 | 0.8 | 0.36 |
| Mg. Stearate | 3 | 1.36 |
| Cabosil | 2 | 0.91 |
| Total | 220 | 100.00 |

The hard gelatin capsule size 00was first filled with the buffered mixture containing the ingredients in table #7 and once the pellets type 1,2 and 3 were formed (5/16″ round deep concave punch, hardness 8-10 SCU) they were filled inside the hard gelatin capsule then finally the immediate release containing nicotine. Snap the cap into the body of the hard gelatin capsule.

### EXAMPLE 4

### Controlled Release Capsule Containing Nicotine Beads

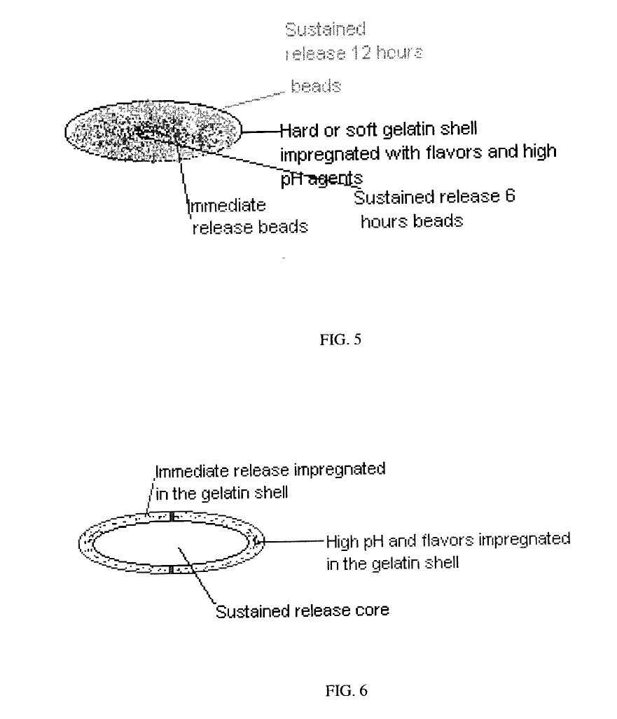

A schematic of the controlled release capsule appears in FIG. 5.

This example provides nicotine beads encapsulated within a capsule impregnated or covered with pH altering and/or flavoring ingredients. Each bead within the capsule contains an inner controlled release core. It is envisioned that the capsule may be placed in the oral cavity and the gelatin layer is dissolved. Subsequently, the capsule may be swallowed with or without the aid of water where the remainder of the capsule and beadlets travel through the stomach, allowing a controlled release of the drug from the beads to maintain plasma levels.

Optionally capsule may be designed to dissolve completely in the oral cavity and allowing the user to swallow the pellets only. With such a capsule, the beads may be enteric coated in order to maintain the beads intact until they reach the intestine. Optionally, each bead may or may not contain an enteric coat of different thickness or a buffer layer on the inner sustained release core in order to have different dissolution profiles e.g. immediate release and 6/12 hours sustained release. Optionally, the beads may contain a buffer layer between the enteric coat and the inner sustained release core. It then provides a rapid dissolution of nicotine from the beads impregnated or coated with nicotine active, thereby resulting in a rapid rise of the drug to therapeutic plasma levels while the formulation is still in the oral cavity.

### EXAMPLE 5

A schematic of the formulation appears in FIG. 6.

Example 5 provides a sustained release nicotine core enrobed within a capsule impregnated or covered with pH altering and/or flavoring ingredients on one side and the other side is impregnated with immediate release nicotine. The enrobing technique could be by dipping the core in a gelatin solution or by wrapping the core with 2 sheets of soft gelatin. The capsule may be placed in the oral cavity for the absorption of the nicotine for immediate release. Subsequently, the capsule may be swallowed with or without the aid of water, the capsule shell may be designed to dissolve completely in the oral cavity and allowing the user to swallow the sustained release core; or if denied, remain intact after hydration in the oral cavity to provide a more slippery surface to facilitate swallowing.

1. An oral controlled release formulation which provides a rapid onset of relief from nicotine cravings following by a sustained period of relief from nicotine cravings comprising:
a) an oral release component for delivering an immediate release of nicotine to the oral and/or buccal cavity; and
b) a gastrointestinal (GI) tract release component for a sustained delivery of nicotine to the stomach and/or intestines.

2. The formulation of claim 1 in a solid form.

3. The formulation of claim 2 wherein said solid form comprises a pill, tablet or capsule wherein:
a) said oral release component is an oral release layer which overlies said GI tract release component; and
b) said GI tract release component is a GI tract release core beneath said oral release layer.

4. The formulation of claim 3 wherein said GI tract release component provides for the immediate release of nicotine into the stomach.

5. The formulation of claim 4 wherein said GI tract release component also provides for nicotine release in the intestines.

6. The formulation of claim 5 wherein the nicotine release in the intestines is sustained release.

7. The formulation of claim 5 wherein the nicotine releases in the intestines is immediate.

8. The formulation of claim 3 wherein little or no nicotine is released in the stomach and the nicotine is released into the intestines.

9. The formulation of claim 1 in a capsule form.

10. The formulation of claim 9 comprising:
a) a capsule which provides for the immediate release of nicotine into the oral and/or buccal cavity; and
b) beads within said capsule which provides for the release of nicotine to the GI tract.

11. The formulation of claim 9 comprising:
a) a capsule; and
b) materials with said capsule to provide for both the immediate oral release of nicotine and the GI tract release of nicotine.

12. The formulation of claim 3 and 11 wherein said oral release component further comprises buffer agents sufficient to modify the pH of saliva into the range of pH 7-pH 10 in order to enhance absorption of nicotine across the mucosa of the oral and buccal cavity.

13. The formulation of claim 3 further comprising a buffer layer overlying or underlying said oral release layer, herein said buffer layer contains buffer agents to be released during the immediate oral release of nicotine so as to optimize the pH of the saliva for optimal absorption of nicotine across the mucosa of the oral and buccal cavities.

14. The formulation of claim 13 further comprising a physical barrier layer interposed the oral release layer and the buffer layer.

15. The formulation of claim 3 further comprising an enteric coating layer interposed the oral release layer and the GI tract release core to provide that after immediate nicotine release in the oral/buccal cavity there is little or no nicotine release in the stomach and a sustained or immediate release of nicotine to the intestines is provided.

16. The formulation of claim 3 wherein said oral release layer is a film comprising:
i) a source of nicotine;
ii) one or more water soluble film-forming polymers;
iii) one or more plasticizers; and
iv) solvent sufficient to form a film of said water soluble polymer and plasticizer.

17. The formulation of claim 16 wherein said water soluble polymers are one or more selected from the group consisting of hydrophilic polymers and polysaccharides, and alkylcellulose polymers.

18. The formulation of claim 17 wherein said hydrophilic polymers and polysaccharides and alkylcelluloses are selected from the group consisting of sodium carboxymethylcellulose, partially cross-linked polyacrylic acid, hydroxyethylcellulose, hydroxypropylcellulose, hydroxypropylmethylcellulose (HPMC), polyethylene oxide, pectin, gelatin, calcium silicate, ethylcellulose, starches, gums and mixtures thereof.

19. The formulation of claim 17 wherein said water soluble polymer is present in said oral release layer in an amount ranging from about 35% to about 95% by weight of said oral release layer.

20. The formulation of claim 19 wherein said water soluble polymer is present in said oral release layer in an amount of from about 75% to about 95% by weight of said oral release layer.

21. The formulation of claim 20 wherein said water soluble polymer is present in an amount of about 80% by weight of said oral release component.

22. The formulation of claim 18 wherein said water soluble polymer is selected from ethylcellulose and HPMC.

23. The formulation of claim 16 wherein said plasticizers are selected from the group consisting of polyethylene glycol, propylene glycol, mineral oils, vegetable oils, triethylacetate, dibutyl sebacate, diethyl phthalate, triethyl citrate, tributyl citrate, triacetin, acetylated monoglycerides, phthalate esters, castor oil and mixtures thereof.

24. The formulation of claim 23 wherein said plasticizer is present in said oral release layer in an amount of from about 1% to about 10% of said water soluble polymers.

25. The formulation of claim 23 wherein said one or more plasticizers are selected form the group consisting of propylene glycol, triethylacetate and triethyl citrate.

26. The formulation of claim 1 wherein said nicotine is present within said formulation as a pharmaceutically acceptable salt of nicotine.

27. The formulation of claim 26 wherein said nicotine salt is present in said oral release layer in a range of 0.1 to 2.0% by weight.

28. The formulation of claim 26 wherein said pharmaceutically acceptable salts are selected from the group consisting of nicotine monotartrate, nicotine bitartrate, nicotine hydrochloride, nicotine dihydrochloride, nicotine sulfate, nicotine zinc chloride monohydrate and nicotine salicylate.

29. The formulation of claim 28 wherein said salt is nicotine bitartrate.

30. The formulation of claim 1 wherein said nicotine is selected from the group consisting of nicotine oil and nicotine ion-exchange resin complexes.

31. The formulation of claim 30 wherein said resin complex is nicotine polacrilex.

32. The formulation of claims 12 and 13 wherein said buffer agents are selected from the group consisting of sodiumcarbonate, sodium bicarbonate, sodium phosphate, calcium carbonate, magnesium carbonate, magnesium hydroxide, aluminum hydroxide and mixtures thereof.

33. The formulation of claim 13 wherein said buffer layer comprises a buffer agent and a film-forming polymer.

34. The formulation of claim 33 wherein said buffer agent is present in an amount of from about 0.01 to about 0.06% by weight of said buffer layer.

35. The formulation of claim 32 wherein said buffer agent is calcium carbonate.

36. The formulation of claim 33 further comprising one or more plasticizers.

37. The formulation of claim 15 wherein said enteric coating layer is a film of a pharmaceutically acceptable acrylic polymer.

38. An oral controlled release form comprising:
i) a GI tract release core comprised of nicotine in a polymer matrix of one or morev selected from hydrophilic polymers and polysaccharides, alkylcellulose polymers, acrylic polymers and mixtures thereof;
ii) an oral release layer overlying said core comprising nicotine in a film of one or more hydrophilic polymers and polysaccharides, alkylcellulose polymers and mixtures thereof; and a buffer layer comprising
iii) one or more buffer agents in a polymer film overlying said oral release layer, said buffer agents being present in an amount sufficient to adjust the pH of saliva to a range optimal for nicotine absorption in the oral/buccal cavity.

39. The formulation of claim 38 further including a physical barrier layer interposed said buffer layer and said oral release layer, said barrier being a polymer film which prevents interaction of nicotine in said oral release layer with buffer agents in said buffer layer.

40. The formulation of claim 38 further comprising an enteric coating layer interposed said core and said oral release layer.

41. A method of providing nicotine replacement therapy to a patient in need thereof using an oral controlled release formulation comprising an oral nicotine release component and a GI tract nicotine release component, which method comprises the steps of:
a) placing said formulation I the oral/buccal cavity of the patient for a time sufficient to deliver nicotine from said oral release component to the oral/buccal cavity thereby providing an initial relief of nicotine craving; and
b) thereafter swallowing said formulation to provide for a sustained release of nicotine from the GI tract release component to the GI tract of said patient thereby providing a sustained relief from nicotine craving.

42. The method of claim 41 wherein said oral release component provides plasma levels of nicotine sufficient to relieve nicotine cravings from about 2 to about 30 minutes following oral administration.

43. The method of claim 41 wherein said GI tract release component provides plasma levels of nicotine sufficient to relieve nicotine cravings for at least about 4 hours after oral administration.

44. The method of claim 41 wherein said oral release component contains about 0.25 mg to about 1.0 mg of nicotine free base.

45. The method of claim 41 wherein said GI tract release component contains about 10-60 mg of nicotine free base.

46. The method of claim 41 wherein said formulation also contains a therapeutically effective amount of an antidepressant.

47. The method of claim 46 wherein said antidepressant is selected from the group consisting of selective serotonin re-uptake inhibitors and tricyclic antidepressants.

48. The method of claim 47 wherein said selective serotonin re-uptake inhibitors are selected from the group consisting of paroxetine, fluoxetine, sertraline, citaloprolam, fluvoxamine, paroxetine and bupropion.

49. The method of claim 47 wherein said tricyclic antidepressants are selected from the group consisting of amitriptylene and nortriptylene.

50. The formulation of claim 1 further comprising a therapeutically effective amount of an antidepressant and or an antianxiolytic.

51. An oral controlled release formulation which provides for immediate release of nicotine active in the stomach and nicotine release in the intestines comprising a two part GI tract release component which comprises:
a) an intestinal tract release core for the immediate or sustained release of nicotine to the intestines; and
b) a stomach release layer for the immediate release of mixture to the stomach.

52. The formulation of claim 51 further comprising an enteric coating layer interposed the intestinal release core and the stomach release layer.
